SEQUENCE LISTING (10 sequences)

SEQ ID NO: 1 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 2 - protein, 595 aa, synthetic construct
  REGION 1..595: note = AMG NL
RPDPKGGNLTPFIHKEGERSLQGILDNLGGRGKKTPGTAAGLFIASPNTENPNYYYTWTRDSALTAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 3 - protein, 595 aa, synthetic construct
  REGION 1..595: note = AMG anPAV498
RNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 4 - protein, 595 aa, synthetic construct
  REGION 1..595: note = AMG JPO-001
ANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 5 - protein, 595 aa, synthetic construct
  REGION 1..595: note = AMG JPO-124
ANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 6 - protein, 595 aa, synthetic construct
  REGION 1..595: note = AMG JPO-172
ANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 7 - protein, 593 aa, Penicillium miczynskii
NPTSKDAKMASYISKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLISHGSKSEAANIMWPIIANDLAYVGQYWNNTGFDLWEEVDGSSFFTLAVQHRSLVQGASLAKKLGKSCPACVSQAPQILCFLQSFWNGNYITANINLDTSRSGIDLNSILGSIHTFDPEASCDDSTFQPCSARALANHKVYVDAFRSIYKVNAGLSNGSAANVGRYPEDVYQGGNPWYLATLASAELLYDALYQWSKIGKLDVTKTSLAFFKDFDMAVKTGTYSAHSSTYKSLTSAIRTYADDFIGLVQDYTPSNGSLAEQYDRNTGIPLSANDLTWSYASFITAIQRRASVVPASWGEKSANVVPTICSASPVTGTYQAAPSVFPTTTGCVPATSIVPITFYLTETTFYGENVYMTGNISALGNWDTNNGFPLTANLYTDSDHLWFASVELVPAGTPFEYKYYKVEPNGTVIWENGENRVYVAPTGCPIQPSQTDVWR

SEQ ID NO: 8 - protein, 590 aa, Penicillium russellii
SKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 9 - protein, 594 aa, Penicillium glabrum
NPFNSLDSFILKEGARSYQGIIDNLGNKGVKAPGTAAGLFVASPNTANPDYFYTWTRDSALTFKCLIDLFDGGSTEFGLKNSELETDIRNYVSSQAVLQNVSNPSGTLEDGTGLGEPKFEVDLNPFTGSWGRPQRDGPALRATALITYTNYLLSQGQKSEAVNIMWPIISNDLAYVGQYWNDTGFDLWEETDGSSFFTLAVQHRALVQGATLAQKLGKSCAACSSQAPQILCFLQSFWNGKYITANINLDTSRTGIDANTLLGSIHTFDPEAACDDSTFQPCSARALANHKVYVDAFRSIYKINSGIAEGSPANVGRYPEDVYQGGNPWYLTTLASAELLYDALYQWNKIGGLDVTETSLAFFKDFHSSVKTGSYSAHSQTYKTLTSAIRTYADGFVGLVQKYTPANGSLAEQYNRNTSVPLSANDLTWSFASFLTAIQRRESIVPGSWGEKSANTVPTTCSASPVTGTYKAATSTFPTSTAGCVPATDVVPITFYLIENTYYGENVYMTGNISALGNWDTSDGLALDAGLYTETDNLWFGTLELVTAGTPFEYKYYKIEPNGTVTIWESGDNRVSVVPTGCPIQPSLHDVWRS

SEQ ID NO: 10 - protein, 591 aa, Rasamsonia emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

(19) United States
(12) Patent Application Publication
(10) Pub. No.: US 2026/0354392 A1
(43) Pub. Date: Nov. 12, 2026

(54) Baking at Low-pH with Thermostable Glucoamylase Variants

(71) Applicant: Novozymes A/S, Bagsvaerd (DK)

(72) Inventors: Katie HARRIS, Franklinton, NC (US); henrik LUNDKVIST, Bagsvaerd (DK)

(73) Assignee: Novozymes A/S, Bagsvaerd (DK)

(21) Appl. No.: 19/210,122

(22) PCT Filed: Nov. 30, 2022
(86) PCT No.: PCT/US2022/080659

§ 371 Date: May 16, 2025

(51) Int. Cl. A21D 8/04

(52) U.S. Cl. CPC A21D 8/042

(57) ABSTRACT

The invention relates to methods of producing a baked or par-baked product, said method comprising: a) providing a dough comprising a mature thermostable variant of a parent glucoamylase at least 70% identical to SEQ ID NO:1, SEQ ID NO:6, SEQ ID NO:7; SEQ ID NO:8 or SEQ ID NO:10 added in an amount of 0.01-12.40 mg enzyme protein (mgEP) per kg flour, wherein said dough has a pH value in the range of 3.0-6.5; and b) baking or par-baking the dough to produce a baked or par-baked product.

### REFERENCE TO SEQUENCE LISTING

This application contains a Sequence Listing in computer readable form, which is incorporated herein by reference.

### FIELD OF THE INVENTION

The invention relates to methods of producing a baked or par-baked product, said method comprising:
a) providing a dough comprising a mature thermostable variant of a parent glucoamylase (AMG) at least 70% identical to SEQ ID NO:1, SEQ ID NO:6, SEQ ID NO:7; SEQ ID NO:8 or SEQ ID NO:10 added in an amount of 0.01-12.40 mg enzyme protein (mgEP) per kg flour, wherein said dough has a pH value in the range of 3.0-6.5; and
b) baking or par-baking the dough to produce a baked or par-baked product.

### BACKGROUND OF THE INVENTION

World-wide, baked products (breads, biscuits, etc.) containing sugar is one of the most popular product segments. The recipe amount of sugar will typically be 1-25% of total flour weight.

However, due to increased market price for sugar, shortage in sugar availability in some parts of the world as well as health-concerns, there is a need for methods of producing baked products that contain a reduced amount of added sugar without sacrificing the quality of the baked product and perhaps even improving it.

WO 2019/238423 (Novozymes A/S, Denmark) discloses methods of producing a dough with a reduced amount of added sugar comprising adding a raw starch degrading alpha-amylase and a glucoamylase to the dough ingredients.

WO 2022/090562 (Novozymes A/S, Denmark) discloses methods of producing a baked or par-baked product with a mature thermostable variant of a parent glucoamylase.

### SUMMARY OF THE INVENTION

Thermostabilized glucoamylase variants show greatly improved performance in freshkeeping or anti-staling of a baked or par-baked product. Another improved performance of thermostabilized variants is that they increase the sweetness or sweet taste of the product, which allows a reduction in the amount of added sugar in traditional recipes. Now, another surprising effect of the thermostabilized glucoamylase variants is shown herein to allow reduced enzyme dosing at reduced dough pH in the range of 3.0-6.5.

Accordingly in a first aspect, the invention relates to methods of producing a baked or par-baked product, said method comprising:
a) providing a dough comprising a mature thermostable variant of a parent glucoamylase having at least 70% sequence identity to SEQ ID NO:1, SEQ ID NO:6, SEQ ID NO:7; SEQ ID NO:8 or SEQ ID NO:10 added in an amount of 0.01-12.40 mg enzyme protein (mgEP) per kg flour, wherein said dough has a pH value in the range of 3.0-6.5; preferably 3.5-6.0; even more preferably 4.0-5.5; and
b) baking or par-baking the dough to produce a baked or par-baked product.

Preferably, the mature thermostable variant of a parent glucoamylase of the invention having at least 71% sequence identity to SEQ ID NO:1, SEQ ID NO:6, SEQ ID NO:7, SEQ ID NO:8 or SEQ ID NO:10, e.g., at least 72%, e.g., at least 73%, e.g., at least 74%, e.g., at least 75%, e.g., at least 76%, e.g., at least 77%, e.g., at least 78%, e.g., at least 79%, e.g., at least 80%, e.g., at least 81%, e.g., at least 82%, e.g., at least 83%, e.g., at least 84%, e.g., at least 85%, e.g., at least 86%, e.g., at least 87%, e.g., at least 88%, e.g., at least 89%, e.g., at least 90%, e.g., at least 91%, e.g., at least 92%, e.g., at least 93%, e.g., at least 94%, e.g., at least 95%, e.g., at least 96%, e.g., at least 97%, e.g., at least 98%, e.g., at least 99% sequence identity to SEQ ID NO:1, SEQ ID NO:6, SEQ ID NO:7, SEQ ID NO:8 or SEQ ID NO:10.

### FIGURES

FIG. 1 shows a multiple alignment of the amino acid sequences of the mature proteins of:
Wild-type AMG from Penicillium oxalicum (PoAMG) of SEQ ID NO:1
PoAMG variant denoted ‘AMG NL’ of SEQ ID NO:2
PoAMG variant denoted ‘AMG anPAV498’ of SEQ ID NO:3
PoAMG variant denoted ‘AMG JPO001’ of SEQ ID NO:4
PoAMG variant denoted ‘AMG JPO124’ of SEQ ID NO:5
PoAMG variant denoted ‘AMG JPO172’ of SEQ ID NO:6
Wild-type AMG from Penicillium miczynskii (PoAMG) of SEQ ID NO:7
Wild-type AMG from Penicillium russellii (PoAMG) of SEQ ID NO:8
Wild-type AMG from Penicillium glabrum (PoAMG) of SEQ ID NO:9

FIG. 2 shows the pH-activity profile of the thermostable glucoamylase variant denoted JPO-172. The pH-activity profile was determined at 40 °C. Each data point represents the average of four measurements and the error bars represent standard deviations. pH 5 is set to 10%. In the profile it can be seen that pH optimum is around pH 5 and that approximately 80% activity is observed from approximately pH 4 to 6.

### DETAILED DESCRIPTION OF THE INVENTION

### Definitions

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the -no brief option) is used as the percent identity and is calculated as follows:

(Identical Residues×100)/(Length of Alignment−Total Number of Gaps in Alignment)

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more amino acids adjacent to and immediately following the amino acid occupying a position. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope, or a binding domain. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Increased strength: The term “increased strength of the dough” is defined herein as the property of a dough that has generally more elastic properties and/or requires more work input to mould and shape compared to a control.

Increased elasticity: The term “increased elasticity of the dough” is defined herein as the property of a dough which has a higher tendency to regain its original shape after being subjected to a certain physical strain compared to a control.

Increased stability of the dough: The term “increased stability of the dough” is defined herein as the property of a dough that is less susceptible to mechanical abuse thus better maintaining its shape and volume and is evaluated by the ratio of height:width of a cross section of a loaf after normal and/or extended proof compared to a control.

Reduced stickiness of the dough: The term “reduced stickiness of the dough” is defined herein as the property of a dough that has less tendency to adhere to surfaces compared to a control, e.g., in the dough production machinery, and it is either evaluated empirically by the skilled test baker or measured by, e.g., a texture analyser (e.g., TAXT2) as known in the art.

Improved extensibility: The term “improved extensibility of the dough” is defined herein as the property of a dough that can be subjected to increased strain or stretching without rupture compared to a control.

Improved machinability: The term “improved machinability of the dough” is defined herein as the property of a dough that is generally less sticky and/or firmer and/or more elastic compared to a control.

Increased volume of the baked product: The term “increased volume of the baked product” is measured as the volume of a given loaf of bread compared to a control. The volume may be determined as known in the art.

Improved crumb structure of the baked product: The term “improved crumb structure of the baked product” is defined herein as the property of a baked product with finer cells and/or thinner cell walls in the crumb and/or more uniform/homogenous distribution of cells in the crumb compared to a control and is usually evaluated visually by the skilled baker or by digital image analysis as known in the art (e. g., C-cell, Calibre Control International Ltd, Appleton, Warrington, UK).

Improved softness of the baked product: The term “improved softness of the baked product” is the opposite of “firmness” and is defined herein as the property of a baked product that is more easily compressed compared to a control and is evaluated either empirically by the skilled test baker or measured by, e.g., a texture analyser (e.g., TAXT2 or TA-XT Plus from Stable Micro Systems Ltd, surrey, UK) as known in the art.

Sensory attributes of the baked products: The sensory attributes may be evaluated using procedures well established in the baking industry, and may include, for example, the use of a panel of trained taste-testers.

Thermostability improvement: The thermostability improvement (Td) in °C is a measure of how much the variants have improved in thermostability over their parent glucoamylase under the same conditions, determined as exemplified herein.

The first aspect of the invention relates to method of producing a baked or par-baked product, said method comprising:
a) providing a dough comprising a mature thermostable variant of a parent glucoamylase at least 70% identical to SEQ ID NO:1, SEQ ID NO:6, SEQ ID NO:7, SEQ ID NO:8 or SEQ ID NO:10; and
b) baking or par-baking the dough to produce a baked or par-baked product.

Other aspects of the invention relate to methods of increasing the sweetness of a baked or par-baked product, for reducing the amount of sugar in the dough in a method of producing a baked or par-baked product and/or for extending the shelf-life of a baked or par-baked product in a method of producing a baked or par-baked product, as well as in methods as defined in the first aspect, whereby the baked or par-baked product after final bake-off has a reduced initial firmness and/or an increased initial elasticity, and/or a reduced increase in firmness and/or a higher elasticity after 1, 7 or 14 days, when cooled to room temperature, packed in a sealed container and stored at room temperature until analysis, compared to a control made without any added glucoamylase.

Preferably, the mature thermostable variant of a parent glucoamylase of the invention which has at least 71% sequence identity to SEQ ID NO:1, SEQ ID NO:6, SEQ ID NO:7, SEQ ID NO:8 or SEQ ID NO:10, e.g., at least 72%, e.g., at least 73%, e.g., at least 74%, e.g., at least 75%, e.g., at least 76%, e.g., at least 77%, e.g., at least 78%, e.g., at least 79%, e.g., at least 80%, e.g., at least 81%, e.g., at least 82%, e.g., at least 83%, e.g., at least 84%, e.g., at least 85%, e.g., at least 86%, e.g., at least 87%, e.g., at least 88%, e.g., at least 89%, e.g., at least 90%, e.g., at least 91%, e.g., at least 92%, e.g., at least 93%, e.g., at least 94%, e.g., at least 95%, e.g., at least 96%, e.g., at least 97%, e.g., at least 98%, e.g., at least 99% sequence identity to SEQ ID NO:1, SEQ ID NO:6, SEQ ID NO:7, SEQ ID NO:8 or SEQ ID NO:10.

### The Dough

As used herein “dough” means any dough used to prepare a baked product, in particular a bread.

According to the present invention, the dough used to prepare a baked product may be made from any suitable dough ingredients comprising flour.

The flour may be from any baking grain known in the art, such as, wheat flour, corn flour, rye flour, barley flour, oat flour, rice flour, sorghum flour, potato flour, soy flour, and any combinations thereof (e.g., wheat flour combined with one of the other flour sources; or rice flour combined with one of the other flour sources).

In a preferred embodiment, the flour is wheat flour.

In a preferred embodiment, at least 10% (w/w) or more of the total flour content is wheat flour, e.g., at least 15% or more of the total flour content is wheat flour, e.g., at least 20% or more of the total flour content is wheat flour, e.g., at least 25% or more of the total flour content is wheat flour, e.g., at least 30% or more of the total flour content is wheat flour, e.g., at least 35% or more of the total flour content is wheat flour, e.g., at least 40% or more of the total flour content is wheat flour, e.g., at least 45% or more of the total flour content is wheat flour, e.g., at least 50% or more of the total flour content is wheat flour, e.g., at least 55% or more of the total flour content is wheat flour, e.g., at least 60% or more of the total flour content is wheat flour, e.g., at least 65% or more of the total flour content is wheat flour, e.g., at least 70% or more of the total flour content is wheat flour, e.g., at least 75% or more of the total flour content is wheat flour, e.g., at least 80% or more of the total flour content is wheat flour, e.g., at least 85% or more of the total flour content is wheat flour, e.g., at least 90% or more of the total flour content is wheat flour, e.g., at least 95% or more of the total flour content is wheat flour, e.g., 100% of total the flour is wheat flour.

The dough of the invention is normally a leavened dough or a dough to be subjected to leavening. The dough may be leavened in various ways, such as by adding dough ingredients such as chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g., a commercially available strain of S. cerevisiae.

The dough of the invention may typically comprise some added sugar as the method according to the invention is able to reduce the amount of added sugar, but normally a partially reduction of sugar is obtained.

In one embodiment, the amount of added sugar is reduced by at least 10% (w/w) compared to the amount of sugar added to a dough in an original recipe, e.g., the amount of added sugar is reduced by at least 20% (w/w) compared to the amount of sugar added to a dough in an original recipe, e.g., the amount of added sugar is reduced by at least 30% (w/w) compared to the amount of sugar added to a dough in an original recipe, e.g., the amount of added sugar is reduced by at least 40% (w/w) compared to the amount of sugar added to a dough in an original recipe, e.g., the amount of added sugar is reduced by at least 50% (w/w) compared to the amount of sugar added to a dough in an original recipe, e.g., the amount of added sugar is reduced by at least 60% (w/w) compared to the amount of sugar added to a dough in an original recipe, e.g., the amount of added sugar is reduced by at least 70% (w/w) compared to the amount of sugar added to a dough in an original recipe, e.g., the amount of added sugar is reduced by at least 80% (w/w) compared to the amount of sugar added to a dough in an original recipe, e.g., the amount of added sugar is reduced by at least 90% (w/w) compared to the amount of sugar added to a dough in an original recipe, e.g., the amount of added sugar is reduced by 100% (w/w) compared to the amount of sugar added to a dough in an original recipe.

The dough may also comprise other conventional dough ingredients, e.g., proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a salt such as sodium chloride, calcium acetate, sodium sulphate, calcium sulphate, diluents such as silica dioxide, and starch of different origins. Still other conventional ingredients include hydrocolloids such as CMC, guar gum, xanthan gum, locust bean gum, etc.

The dough ingredients may typically comprise fat (triglyceride) and/or oil and/or shortenings, in particular oil such as sunflower oil or rapeseed oil.

In a preferred embodiment, no emulsifier is added to the dough of the invention, preferably no SSL is added to the dough of the invention.

In a preferred embodiment, the pH of the dough of the invention is adjusted to the range of 3.0-6.5; preferably 3.5-6.0; even more preferably 4.0-5.5, by addition of an food acceptable acid to the dough, preferably an organic acid, such as, acetic acid or citric acid, and most preferably the pH of the dough is adjusted by the addition of vinegar to the dough.

The dough may be prepared applying any conventional mixing process, such as the continuous mix process, straight-dough process, or the sponge and dough method.

The present invention is particularly useful for preparing dough and baked products in industrialized processes in which the dough used to prepare the baked products are prepared mechanically using automated or semi-automated equipment.

The process of preparing bread generally involves the sequential steps of dough making, sheeting or dividing, shaping or rolling, and proofing the dough, which steps are well known in the art.

As used herein, “baked product” means any kind of baked product including bread types such as pan bread, toast bread, open bread, pan bread with and without lid, buns, Fino bread, Hammam bread, Samoli bread, baguettes, brioche hamburger buns, rolls, brown bread, whole meal bread, rich bread, bran bread, flat bread, tortilla, biscuits, and any variety thereof. According to the present invention, the baked product may also be a cake or any patisserie product as known in the art.

### Raw Starch Degrading Alpha-Amylase

As used herein, a “raw starch degrading alpha-amylase” refers to an enzyme that can directly degrade raw starch granules below the gelatinization temperature of starch.

Examples of raw starch degrading alpha-amylases include the ones disclosed in WO 2005/003311, U.S. Patent Publication no. 2005/0054071, and U.S. Pat. No. 7,326,548. Examples also include those enzymes disclosed in Tables 1 to 5 of the examples in U.S. Pat. No. 7,326,548, in U.S. Patent Publication no. 2005/0054071 (Table 3 on page 15), as well as the enzymes disclosed in WO 2004/020499 and WO 2006/06929 and WO 2006/066579.

In one embodiment, the raw starch degrading alpha-amylase is a GH13_1 amylase.

In one embodiment, the raw starch degrading alpha-amylase enzyme has at least 70%, e.g., at least 71%, e.g., at least 72%, e.g., at least 73%, e.g., at least 74%, e.g., at least 75%, e.g., at least 76%, e.g., at least 77%, e.g., at least 78%, e.g., at least 79%, e.g., at least 80%, e.g., at least 81%, e.g., at least 82%, e.g., at least 83%, e.g., at least 84%, e.g., at least 85%, e.g., at least 86%, e.g., at least 87%, e.g., at least 88%, e.g., at least 89%, e.g., at least 90%, e.g., at least 91%, e.g., at least 92%, e.g., at least 93%, e.g., at least 94%, e.g., at least 95%, e.g., at least 96%, e.g., at least 97%, e.g., at least 98%, e.g., at least 99% sequence identity to the raw starch degrading alpha-amylase shown in EP U.S. Pat. No. 2,981,170 (Novozymes A/S).

In one embodiment, the raw starch degrading alpha-amylase according to the invention may be added to flour or dough in an amount of 0.01-10 mg enzyme protein per kg flour, e.g., in an amount of 0.1-5 mg enzyme protein per kg flour.

### Glucoamylases

Glucoamylases are also called amyloglucosidases, and Glucan 1,4-alpha-glucosidase (EC 3.2.1.3), more commonly they are referred to as AMGs.

According to the present invention, different types of amyloglucosidases may be used as parent for the generation of a thermostable amyloglucosidase variant, e.g., the amyloglucosidase may be a polypeptide that is encoded by a DNA sequence that is found in a fungal strain of Aspergillus, Rhizopusor, Talaromyces or Penicillium; preferably the DNA sequence that is found in a fungal strain of Penicillium, even more preferably the DNA sequence that is found in a fungal strain of Penicillium oxysporum, Penicillium oxalicum, Penicillium miczynskii, Penicillium russellii or Penicillium glabrum. Preferably, the parent glucoamylase is from a species of Penicillium, preferably from Penicillium oxicalum, Penicillium miczynskii, Penicillium russellii or Penicillium glabrum.

Examples of other suitable fungi include Aspergillus niger, Aspergillus awamori, Aspergillus oryzae, Rhizopus delemar, Rhizopus niveus, Rhizopus oryzae and Talaromyces emersonii.

Below is shown the %-identity between the AMG amino acid sequences aligned in FIG. 1, and also provided in the sequence list:

| | | | | | | | | | |
|---|---|---|---|---|---|---|---|---|---|
| P_oxalicum | 100.00 | 99.83 | 98.99 | 98.82 | 96.64 | 95.97 | 77.07 | 77.12 | 74.32 |
| AMG_NL | 99.83 | 100.00 | 99.16 | 98.99 | 96.81 | 96.13 | 77.07 | 77.12 | 74.32 |
| AMG_anPAV498 | 98.99 | 99.16 | 100.00 | 99.83 | 97.65 | 96.97 | 76.73 | 76.95 | 73.82 |
| AMG_JPO001 | 98.82 | 98.99 | 99.83 | 100.00 | 97.82 | 97.14 | 76.73 | 76.95 | 73.82 |
| AMG_JPO124 | 96.64 | 96.81 | 97.65 | 97.82 | 100.00 | 99.33 | 77.07 | 77.12 | 74.32 |
| AMG_JPO172 | 95.97 | 96.13 | 96.97 | 97.14 | 99.33 | 100.00 | 76.73 | 76.78 | 73.99 |
| P_miczynskii | 77.07 | 77.07 | 76.73 | 76.73 | 77.07 | 76.73 | 100.00 | 94.75 | 80.51 |
| P_russellii | 77.12 | 77.12 | 76.95 | 76.95 | 77.12 | 76.78 | 94.75 | 100.00 | 79.66 |
| P_glabrum | 74.32 | 74.32 | 73.82 | 73.82 | 74.32 | 73.99 | 80.51 | 79.66 | 100.00 |

In one embodiment, the glucoamylase according to the invention may be added to flour or dough in an amount 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

Thermostable variants of the PoAMG have been generated (see table 2 below). In a preferred embodiment, the mature thermostable glucoamylase variant of the invention comprises one or more or all of the combinations of amino acid substitutions listed in table 2 below.

In a preferred embodiment, the mature variant of the invention comprises at least one amino acid modification in one or more or all of the positions corresponding to positions 1, 2, 4, 6, 7, 11, 31, 34, 65, 79, 103, 132, 327, 445, 447, 481, 566, 568, 594 and 595 in SEQ ID NO:1; preferably the at least one amino acid modification comprises a substitution in one or more or all of the positions corresponding to positions 1, 2, 4, 11, 65, 79 and 327 in SEQ ID NO:1, preferably the at least one amino acid modification comprises a substitution in one or more or all of the positions corresponding to R1A, P2N, P4S, P11F, T65A, K79V and Q327F in SEQ ID NO:1; or preferably the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1, preferably the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO:1; or preferably the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1, preferably the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.

The thermostability improvements (Td) of the variants in table 2 are listed in Table 3, where the Td of the PoAMG variant denoted “anPAV498” (the parent) was set to zero. In a preferred embodiment, the mature thermostable variant of the invention has a thermostability improvement (Td) over its parent of at least 3 °C, preferably at least 4 °C, 5 °C, 6 °C, 7 °C or 8 °C, preferably determined as exemplified herein.

In another preferred embodiment, the mature thermostable variant of the invention has a relative activity at 91 °C of at least 150, preferably at least 200, more preferably at least 250, most preferably at least 300 compared to its parent.

Preferably, the mature thermostable variant glucoamylase enzyme is comprised in the dough in an amount of 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

### Amylases

Alpha-Amylases (alpha-1,4-glucan-4-glucanohydrolases, EC. 3.2.1.1) constitute a group of enzymes which catalyze hydrolysis of starch and other linear and branched 1,4-glucosidic oligo- and polysaccharides.

A number of alpha-amylases are referred to as Termamyl™ and “Termamyl™-like alpha-amylases” and are known from, e.g., WO 90/11352, WO 95/10603, WO 95/26397, WO 96/23873 and WO 96/23874.

Another group of alpha-amylases are referred to as Fungamyl™ and “Fungamyl™-like alpha-amylases”, which are alpha-amylases related to the alpha-amylase derived from Aspergillus oryzae disclosed in WO 01/34784.

Suitable commercial alpha-amylase compositions according to the present invention include, e.g., BAKEZYME P 300 (available from DSM) and FUNGAMYL 2500 SG, FUNGAMYL 4000 BG, FUNGAMYL 4000 SG, FUNGAMYL 800 L, FUNGAMYL ULTRA BG and FUNGAMYL ULTRA SG (available from Novozymes A/S).

In one embodiment, the alpha-amylase according to the invention may be added to flour or dough in an amount of 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

### Additional Enzymes

Optionally, one or more additional enzymes, such as alpha-amylase, maltogenic amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase may be used together with the enzyme composition according to the invention.

The additional enzyme(s) may be of any origin, including mammalian, plant, and microbial (bacterial, yeast or fungal) origin.

The maltogenic alpha-amylase (EC 3.2.1.133) may be from Bacillus. A maltogenic alpha-amylase from B. stearothermophilus strain NCIB 11837 is commercially available from Novozymes A/S under the tradename Novamyl®.

The maltogenic alpha-amylase may also be a variant of the maltogenic alpha-amylase from B. stearothermophilus as disclosed in, e.g., WO 99/43794; WO 2006/032281; or WO 2008/148845, e.g., Novamyl® 3D.

An anti-staling amylase for use in the invention may also be an amylase (glucan 1,4-alpha-maltotetrahydrolase (EC 3.2.1.60)) from Pseudomonas saccharophilia or variants thereof, such as any of the amylases disclosed in WO 99/50399, WO 2004/111217 or WO 2005/003339.

The glucose oxidase may be a fungal glucose oxidase, in particular an Aspergillus niger glucose oxidase (such as GLUZYMER, available from Novozymes A/S).

The xylanase which may be of microbial origin, e.g., derived from a bacterium or fungus, such as a strain of Aspergillus, in particular, A. aculeatus, A. niger, A. awamori, or A. tubigensis, from a strain of Trichoderma, e.g., T. reesei, or from a strain of Humicola, e.g., H. insolens.

Suitable commercially available xylanase preparations for use in the present invention include PANZEA BG, PENTOPAN MONO BG and PENTOPAN 500 BG (available from Novozymes A/S), GRINDAMYL POWERBAKE (available from Danisco), and BAKEZYME BXP 5000 and BAKEZYME BXP 5001 (available from DSM).

The protease may be from Bacillus, e.g., B. amyloliquefaciens. A suitable protease may be Neutrase® available from Novozymes A/S.

The phospholipase may have phospholipase A1, A2, B, C, D or lysophospholipase activity; it may or may not have lipase activity. It may be of animal origin, e.g., from pancreas, snake venom or bee venom, or it may be of microbial origin, e.g., from filamentous fungi, yeast or bacteria, such as Aspergillus or Fusarium, e.g., A. niger, A. oryzae or F. oxysporum. A preferred lipase/phospholipase from Fusarium oxysporum is disclosed in WO 98/26057. Also, the variants described in WO 00/32758 may be used.

Suitable phospholipase compositions are LIPOPAN F, LIPOPAN XTRA, and LIPOPAN MAX (available from Novozymes A/S) or PANAMORE GOLDEN and PANAMORE SPRING (available from DSM).

Preferably, the one or more additional enzyme is added in an amount of 0.01-1,000 mg enzyme protein (mgEP) per kg flour, preferably in an amount of 0.01-500 mg enzyme protein (mgEP) per kg flour, even more preferably in an amount of 0.1-100 mg enzyme protein (mgEP) per kg flour.

### Enzyme Compositions

The mature thermostable variant glucoamylase of the invention as well as any additional enzyme(s) may be added to flour or dough in any suitable form, such as, e.g., in the form of a liquid, in particular a stabilized liquid, or it may be added to flour or dough as a substantially dry powder or granulate.

Granulates may be produced, e.g., as disclosed in U.S. Pat. Nos. 4,106,991 and 4,661,452. Liquid enzyme preparations may, for instance, be stabilized by adding a sugar or sugar alcohol or lactic acid according to established procedures. Other enzyme stabilizers are well-known in the art.

The enzyme(s) may be added to the bread dough ingredients in any suitable manner, such as individual components (separate or sequential addition of the enzymes) or addition of the enzymes together in one step or one composition.

### Bread Properties

Organoleptic qualities or sensory attributes of the bread may be measured as known in the art. The properties of the bread may be referred to herein as sensory attributes, which include anti-staling (bread crumb firmness/hardness), crumb properties and mouth feel, or more precisely, the attributes of bread as detected in the mouth during eating (e.g., bread softness/resistance to first bite, crumb moistness, crumb chewiness and gumminess, and crumb smoothness and melting properties).

In one embodiment, the sensory attribute of the baked product is an increased sweetness by using the enzyme solution according to the invention.

In one embodiment, the sensory attribute of the baked product is an increased crumb sweetness by using the enzyme solution according to the invention.

In a preferred embodiment of the invention, the baked or par-baked product after final bake-off has a reduced initial firmness and/or an increased initial elasticity, and/or a reduced increase in firmness and/or a higher elasticity after 1, 7 or 14 days, when cooled to room temperature, packed in a sealed container and stored at room temperature until analysis, compared to a control made without any added glucoamylase.

In another preferred embodiment, the baked or par-baked product after final bake-off has at least the same sweetness or sweet taste as a control product made with double the amount of the mature glucoamylase the amino acid sequence of which is shown in SEQ ID NO:10, preferably determined as exemplified herein; preferably the baked or par-baked product after final bake-off has a higher sweetness or more sweet taste than a control product made with double the amount of the mature glucoamylase the amino acid sequence of which is shown in SEQ ID NO:10, preferably determined as exemplified herein.

The invention described and claimed herein is not to be limited in scope by the specific embodiments herein disclosed, since these embodiments are intended as illustrations of several aspects of the invention. Any equivalent embodiments are intended to be within the scope of this invention as well as combinations of one or more of the embodiments.

Various references are cited herein, the disclosures of which are incorporated by reference in their entireties. The present invention is further described by the following example which should not be construed as limiting the scope of the invention.

### EXAMPLES

### Example 1: Construction of PoAMG Libraries

PoAMG libraries were constructed as follows:

A forward or reverse primer having NNK or desired mutation(s) at target site(s) with 15 bp overlaps each other were designed. Inverse PCR, which means amplification of entire plasmid DNA sequences by inversely directed primers, were carried out with appropriate template plasmid DNA (e.g., plasmid DNA containing JPO-0001 gene) by the following conditions. The resultant PCR fragments were purified by QIAquick Gel extraction kit [QIAGEN], and then introduced into Escherichia coli ECOS Competent E. coli DH5α [NIPPON GENE CO., LTD.]. The plasmid DNAs were extracted from E. coli transformants by MagExtractor plasmid extraction kit [TOYOBO], and then introduced into A. niger competent cells.

### PCR Reaction Mix:

PrimeSTAR Max DNA polymerase [TaKaRa]

| | |
|---|---|
| Total | 25 μl |

| | | |
|---|---|---|
| 1.0 | μl | Template DNA (1 ng/μl) |
| 9.5 | μl | H₂O |
| 12.5 | μl | 2x PrimeSTAR Max pre-mix |
| 1.0 | μl | Forward primer (5 μM) |
| 1.0 | μl | Reverse primer (5 μM) |

### PCR Program:

98 °C/2 min
25× (98 °C/10 sec, 60 °C/15 sec, 72 °C/2 min)
10 °C/hold

### Example 2: Screening for Better Thermostability

B. subtilis libraries constructed as in EXAMPLE 1 were fermented in either 96-well or 24-well MTP containing COVE liquid medium (2.0 g/L sucrose, 2.0 g/L iso-maltose, 2.0 g/L maltose, 4.9 mg/L, 0.2 ml/L 5 N NaOH, 10 ml/L COVE salt, 10 ml/L 1 M acetamide), 32 °C for 3 days. Then, AMG activities in culture supernatants were measured at several temperatures by pNPG assay described as follows.

pNPG Thermostability Assay:

The culture supernatants containing desired enzymes was mixed with same volume of pH 5.0 200 mM NaOAc buffer. Twenty microliter of this mixture was dispensed into either 96-well plate or 8-strip PCR tube, and then heated by thermal cycler at various temperatures for 30 min. Those samples were mixed with 10 μl of substrate solution containing 0.1% (w/v) pNPG [wako] in pH 5.0 200 mM NaOAc buffer and incubated at 70 °C for 20 min for enzymatic reaction. After the reaction, 60 μl of 0.1 M Borax buffer was added to stop the reaction. Eighty microliter of reaction supernatant was taken out and its OD₄₀₅ value was read by photometer to evaluate the enzyme activity.

TABLE 1a
Lists of the relative activity of PoAMG variants
when compared with their parent anPAV498 or
JPO-0001 (anPAV498 w. leader-/propeptide)

| Name | Relative activity of 80 °C/75 °C (%) |
|---|---|
| anPAV498 | 17% |
| JPO-004 | 32% |
| JPO-005 | 15% |
| JPO-006 | 16% |
| JPO-007 | 3% |
| Name | Relative activity of 80 °C/75 °C (%) |
| AnPav498 | 13% |
| JPO-009 | 16% |
| JPO-011 | 15% |
| JPO-012 | 15% |
| JPO-013 | 17% |
| JPO-020 | 20% |
| Name | Relative activity of 80 °C/70 °C (%) |
| JPO-001 | 10% |
| JPO-004 | 29% |
| JPO-009 | 13% |
| JPO-014 | 21% |
| JPO-020 | 16% |
| JPO-021 | 30% |
| JPO-052 | 33% |
| Name | Relative activity of 79 °C/70 °C (%) |
| JPO-001 | 23% |
| JPO-021 | 46% |
| JPO-022 | 39% |
| JPO-023 | 44% |
| JPO-025 | 51% |
| JPO-027 | 49% |
| JPO-029 | 37% |
| Name | Relative activity of 77 °C/70 °C (%) |
| JPO-001 | 72% |
| JPO-029 | 82% |
| JPO-047 | 80% |
| JPO-048 | 90% |
| JPO-049 | 84% |
| JPO-050 | 86% |
| JPO-064 | 87% |
| name | Relative activity of 79 °C/77 °C (%) |
| JPO-001 | 36% |
| JPO-029 | 51% |
| JPO-047 | 45% |
| JPO-048 | 81% |
| JPO-049 | 53% |
| JPO-050 | 58% |
| JPO-064 | 65% |
| Name | Relative activity of 79 °C/77 °C (%) |
| JPO-001 | 41% |
| JPO-021 | 60% |
| JPO-022 | 48% |
| JPO-023 | 57% |
| JPO-025 | 56% |
| JPO-027 | 64% |
| JPO-029 | 66% |
| JPO-047 | 50% |
| JPO-048 | 72% |
| JPO-051 | 82% |
| JPO-058 | 73% |
| JPO-062 | 72% |
| JPO-063 | 85% |
| JPO-064 | 83% |

TABLE 1b
Lists of the relative activity of PoAMG variants
when compared with their parent JPO-022

| | Relative activity of |
|---|---|
| Name | 77 °C/70 °C (%) |
| JPO-022 | 60% |
| JPO-027 | 67% |
| JPO-042 | 8% |
| JPO-044 | 86% |
| JPO-045 | 67% |
| JPO-046 | 48% |
| JPO-022 | 76% |
| JPO-023 | 75% |
| JPO-025 | 80% |
| JPO-027 | 84% |
| JPO-058 | 92% |
| JPO-059 | 88% |
| JPO-060 | 86% |
| JPO-061 | 83% |
| JPO-062 | 87% |
| | Relative activity of |
| Name | 79 °C/77 °C (%) |
| JPO-022 | 49% |
| JPO-023 | 51% |
| JPO-025 | 52% |
| JPO-027 | 58% |
| JPO-058 | 69% |
| JPO-059 | 36% |
| JPO-060 | 41% |
| JPO-061 | 44% |
| JPO-062 | 57% |

TABLE 1c
List of the relative activity of PoAMG variants
when compared with their parent JPO-063

| | Relative activity of |
|---|---|
| Name | 79 °C/77 °C (%) |
| JPO-063 | 91% |
| JPO-066 | 96% |
| JPO-071 | 89% |
| JPO-072 | 84% |
| JPO-074 | 103% |
| JPO-075 | 86% |
| JPO-076 | 92% |
| JPO-077 | 95% |
| JPO-078 | 88% |
| JPO-079 | 100% |
| | Relative activity of |
| Name | 84 °C/80 °C (%) |
| JPO-063 | 16% |
| JPO-065 | 26% |
| JPO-067 | 21% |
| JPO-070 | 12% |
| JPO-071 | 13% |
| JPO-074 | 32% |
| JPO-081 | 17% |
| JPO-082 | 24% |
| JPO-083 | 46% |
| JPO-084 | 26% |
| JPO-044 | 37% |
| | Relative activity of |
| Name | 82 °C/70 °C (%) |
| JPO-063 | 21% |
| JPO-093 | 43% |
| JPO-081 | 25% |
| JPO-088 | 39% |
| JPO-094 | 38% |
| JPO-096 | 38% |
| JPO-106 | 53% |
| | Relative activity of |
| Name | 83 °C/80 °C (%) |
| JPO-063 | 46% |
| JPO-051 | 44% |
| JPO-096 | 64% |
| JPO-106 | 88% |
| JPO-110 | 81% |
| JPO-111 | 100% |
| JPO-112 | 86% |
| JPO-113 | 83% |
| JPO-114 | 47% |
| JPO-115 | 90% |

TABLE 1d
List of the relative activity of PoAMG variants
when compared with their parent JPO-096

| | Relative activity of |
|---|---|
| Name | 83 °C/70 °C (%) |
| JPO-082 | 53% |
| JPO-088 | 70% |
| JPO-091 | 69% |
| JPO-092 | 65% |
| JPO-093 | 62% |
| JPO-094 | 74% |
| JPO-095 | 69% |
| JPO-096 | 67% |
| JPO-097 | 65% |
| JPO-098 | 65% |
| | Relative activity of |
| Name | 83 °C/80 °C (%) |
| JPO-051 | 20% |
| JPO-096 | 43% |
| JPO-109 | 51% |
| JPO-126 | 33% |
| JPO-129 | 48% |
| JPO-130 | 18% |
| JPO-131 | 51% |
| JPO-132 | 34% |

TABLE 1e
List of the relative activity of PoAMG variants
when compared with their parents JPO-129

| | Relative activity of |
|---|---|
| Name | 84 °C/80 °C (%) |
| JPO-129 | 62% |
| JPO-156 | 51% |
| JPO-160 | 34% |
| JPO-161 | 41% |
| JPO-162 | 49% |
| JPO-163 | 21% |
| JPO-164 | 57% |
| JPO-165 | 77% |

TABLE 1f
List of the relative activity of PoAMG variants
when compared with their parent JPO-166

| | Relative activity of |
|---|---|
| Name | 84 °C/75 °C (%) |
| JPO-166 | 19% |
| JPO-167 | 66% |
| JPO-168 | 58% |
| JPO-169 | 53% |
| JPO-171 | 47% |
| JPO-172 | 98% |

TABLE 2
Amino acid substitutions in the variants of the PoAMG mature sequence

| Name | Amino acid substitutions |
|---|---|
| PoAMG | The wildtype mature AMG from Penicillium (SEQ ID NO: 1) |
| AMG NL | K79V |
| anPAV498 | P2N P4S P11F T65A K79V Q327F |
| JPO-001 | R1A P2N P4S P11F T65A K79V Q327F |
| JPO-018 | D75N R77D A78Q |
| JPO-019 | D75S R77G A78W V79D F80Y |
| JPO-023 | R1A K34Y S103N |
| JPO-024 | R1A K34Y D445N V447S |
| JPO-025 | R1A K34Y Y504T |
| JPO-026 | R1A S103N D445N V447S |
| JPO-027 | R1A S103N Y504T |
| JPO-028 | R1A D445N V447S Y504T |
| JPO-029 | R1A K34Y S103N D445N V447S |
| JPO-044 | R1A K34Y S103N D445N V447S E501V Y504T |
| JPO-047 | R1A K34Y S103N Y504T |
| JPO-048 | R1A K34Y S103N D445N V447S D566T |
| JPO-049 | R1A K34Y S103N Q594R F595S |
| JPO-050 | R1A K34Y S103N Y504T Q594R F595S |
| JPO-051 | R1A K34Y S103N D445N V447S Y504T Q594R F595S |
| JPO-052 | R1A S105L |
| JPO-053 | R1A S105E |
| JPO-055 | R1A A132R |
| JPO-058 | R1A K34Y S105L Y504T Q594R F595S |
| JPO-059 | R1A K34Y S103N S105L Y504T Q594R F595S |
| JPO-060 | R1A K34Y S103N S105L Y504T Q594R F595S |
| JPO-061 | R1A K34Y S103N S105L Y504T D566T Q594R F595S |
| JPO-062 | R1A K34Y S103N S105L D445N V447S Y504T D566T Q594R F595S |
| JPO-063 | R1A K34Y S103N S105L A132R D445N V447S Y504T D566T Q594R F595S |
| JPO-064 | R1A K34Y S103N S105L D445N V447S D566T Q594R F595S |
| JPO-065 | R1A K34Y S103N S105L A132R D445N V447S E501V Y504T D566T Q594R |
| | F595S |
| JPO-066 | R1A K34Y S103N A132R D445N V447S Y504T D566T Q594R F595S |
| JPO-069 | R1A K34Y S103N S105L A132R D445N V447S Y504T D566T V592T |
| JPO-071 | R1A G6S G7T K34Y S103N S105L A132R D445N V447S Y504T D566T Q594R |
| | F595S |
| JPO-074 | R1A K34Y S103N P107L A132R D445N V447S Y504T D566T Q594R F595S |
| JPO-083 | R1A G6S G7T K34Y S103N P107L A132R D445N V447S Y504T D566T Q594R |
| | F595S |
| JPO-084 | R1A G6S G7T K34Y S103N P107L A132R D445N V447S Y504T D566T V592T |
| | Q594R F595S |
| JPO-091 | R1A G6S R7T K34Y S103N P107L A132P D445N V447S Y504T D566T Q594R |
| | F595S |
| JPO-092 | R1A G6S G7T K34Y S103N P107L A132R D445N V447S Y504T D566T T568V |
| | Q594R F595S |
| JPO-093 | R1A G6S G7T K34Y S103N P107L A132P D445N V447S Y504T D566T T568V |
| | Q594R F595S |
| JPO-094 | R1A G6S G7T K34Y S103N P107L A132R D445N V447S S481P Y504T D566T |
| | Q594R F595S |
| JPO-095 | R1A G6S G7T K34Y S103N P107L A132R D445N V447S S481P Y504T D566T |
| | T568V Q594R F595S |
| JPO-096 | R1A G6S G7T K34Y S103N P107L A132P D445N V447S D566T T568V Q594R |
| | F595S |
| JPO-097 | R1A G6S G7T K34Y S103N P107L T110W A132P D445N V447S Y504T D566T |
| | T568V Q594R F595S |
| JPO-098 | R1A G6S G7T K34Y E50R S103N P107L A132P D445N V447S Y504T D566T |
| | T568V Q594R F595S |
| JPO-105 | R1A G6S G7T K34Y S103N P107L A132P D445N V447S E501V Y504T |
| JPO-106 | R1A G6S G7T R31F K34Y S103N P107L A132P D445N V447S Y504T D566T |
| | T568V Q594R F595S |
| JPO-108 | R1A G6S G7T R31F K34Y S103N P107L A132P D445N V447S S481P Y504T |
| | D566T T568V Q594R F595S |
| JPO-109 | R1A G6S G7T K34Y E50R S103N P107L A132P D445N V447S S481P Y504T |
| | D566T T568V Q594R F595S |
| JPO-111 | R1A G6S G7T R31F K34Y S103N P107L A132P D445N V447S S481P E501V |
| | Y504T D566T T568V Q594R F595S |
| JPO-112 | R1A G6S G7T R31F K34Y S103N P107L A132P D445N V447S S481P D566T |
| | T568V Q594R F595S |
| JPO-114 | R1A K34Y D75N R77D A78Q S103N R138L D445N V447S Y504T Q594R F595S |
| JPO-115 | R1A G6S G7T R31F K34Y D75N R77D A78Q S103N P107L A132P D445N V447S |
| | S481P Y504T D566T T568V Q594R F595S |
| JPO-124 | R1A G6S G7T R31F K34Y S103N A132P D445N V447S S481P D566T T568V |
| | Q594R F595S |
| JPO-125 | R1A G6S G7T K34Y E50R S103N A132P D445N V447S S481P D566T T568V |
| | Q594R F595S |
| JPO-126 | R1A R31F K34Y D75N R77D A78Q S103N R138L D445N V447S Y504T Q594R |
| | F595S |
| JPO-127 | R1A K34Y D75N R77D A78Q S103N R138L D445N V447S Q594R F595S |
| JPO-128 | R1A G6S G7T R31F K34Y S103N A132P D445N V447S |
| JPO-129 | R1A G6S G7T R31F K34Y E50R S103N A132P D445N V447S S481P D566T |
| | T568V Q594R F595S |
| JPO-130 | R1A K34Y E50R D75N R77D A78Q S103N R138L D445N V447S Q594R F595S |
| JPO-131 | R1A G6S G7T R31F K34Y E50R D75N R77D A78Q S103N A132P D445N V447S |
| | S481P D566T Q594R F595S |
| JPO-132 | R1A R31F K34Y E50R D75N R77D A78Q S103N R138L D445N V447S Q594R |
| | F595S |
| JPO-133 | R1A G6S G7T R31F K34Y E50R D75N R77D A78Q S103N A132P R138L D445N |
| | V447S S481P D566T Q594R F595S |
| JPO-138 | R1A R135S |
| JPO-143 | R1A G6S G7T R31F K34Y E50R S103N A132P D445N V447S S481P E501L |
| | D566T T568V Q594R F595S |
| JPO-154 | R1A G6S G7T R31F K34Y S103N A132P R138G D445N V447S S481P D566T |
| | T568V Q594R F595S |
| JPO-155 | R1A G6S G7T R31F K34Y S103N A132P R138L D445N V447S S481P D566T |
| | T568V Q594R F595S |
| JPO-156 | R1A G6S G7T R31F K34Y S103N A132P R138P D445N V447S S481P D566T |
| | T568V Q594R F595S |
| JPO-167 | R1A G6S G7T R31F K34Y E50R S103N A132P S379P D445N V447S S481P |
| | E501A D566T T568V Q594R F595S |
| JPO-168 | R1A G6S G7T R31F K34Y E50R S103N A132P D445N V447S S481P T484P |
| | E501A D566T T568V Q594R F595S |
| JPO-169 | R1A G6S G7T R31F K34Y E50R S103N A132P D445N V447S S481P E501A |
| | N539P D566T T568V Q594R F595S |
| JPO-171 | R1A G6S G7T R31F K34Y E50R S103N A132P S379P D445N V447S S481P |
| | T484P E501A D566T T568V Q594R F595S |
| JPO-172 | R1A G6S G7T R31F K34Y E50R S103N A132P D445N V447S S481P T484P |
| | E501A N539P D566T T568V Q594R F595S |

### Example 3: Fermentation of the Aspergillus niger

Aspergillus niger strains were fermented on a rotary shaking table in 500 ml baffled flasks containing 100 ml MU1 with 4 ml 50% urea at 220 rpm, 30 °C. The culture broth was centrifuged (10,000×g, 20 min) and the supernatant was carefully decanted from the precipitates.

### Example 4: Purification of PoAMG (JPO-001) Variants

PoAMG variants were purified by cation exchange chromatography. The peak fractions of each were pooled individually and dialyzed against 20 mM sodium acetate buffer pH 5.0, and then the samples were concentrated using a centrifugal filter unit (Vivaspin Turbo 15, Sartorius). Enzyme concentrations were determined by A280 value.

### Example 5: Thermostability Determination (TSA)

Purified enzyme was diluted with 50 mM sodium acetate buffer pH 5.0 to 0.5 mg/ml and mixed with equal volume of SYPRO Orange (Invitrogen) diluted with Milli-Q water. Eighteen ul of mixture solution were transfer to LightCycler 480 Multiwell Plate 384 (Roche Diagnostics) and the plate was sealed.

### Equipment Parameters of TSA:

Apparatus: LightCycler 480 Real-Time PCR System (Roche Applied Science)
Scan rate: 0.02 °C/sec
Scan range: 37-96 °C
Integration time: 1.0 sec
Excitation wave length 465 nm
Emission wave length 580 nm

The obtained fluorescence signal was normalized into a range of 0 and 1. The Td was defined as the temperature at which the signal intensity was 0.5. The thermostability improvements are listed in Table 3 with Td of the PoAMG variant denoted anPAV498 as 0.

### Example 6: PoAMG Activity Assay

Maltodextrin (DE11) assay by GOD-POD method

### Substrate Solution

30 g maltodextrin (pindex #2 from MATSUTANI chemical industry Co., Ltd.) 100 ml 120 mM sodium acetate buffer, pH 5.0 Glucose CII test kit (Wako Pure Chemical Industries, Ltd.)

Twenty ul of enzyme samples were mixed with 100 ul of substrate solution and incubated at set temperatures for 2 hours. The samples were cooled down on the aluminum block for 3 min then 10 ul of the reaction solution was mixed with 590 ul of 1 M Tris-HCl PH 8.0 to stop reaction. Ten ul of the solution was mixed with 200 ul of the working solution of the test kit then stand at room temperature for 15 min. The absorbance at A505 was read. The activities are listed in Table 3 as relative activity of the PoAMG variant denoted anPAV498.

TABLE 3

| | Td improvement [°C] | Activity at 91 °C |
|---|---|---|
| Variant | (pH 5.0, anPAV498 as 0) | (anPAV498 as 100) |
| anPAV498 | — | 100 |
| JPO-001 | 1.0 | 94 |
| JPO-004 | 2.2 | — |
| JPO-009 | 0.7 | — |
| JPO-013 | 1.5 | — |
| JPO-014 | 2.3 | — |
| JPO-020 | 1.4 | 74 |
| JPO-021 | 2.5 | 113 |
| JPO-052 | 2.6 | 85 |
| JPO-053 | 0.2 | 71 |
| JPO-055 | 1.6 | 85 |
| JPO-023 | 3.6 | — |
| JPO-024 | 2.5 | — |
| JPO-025 | 3.4 | — |
| JPO-027 | 2.9 | — |
| JPO-029 | 3.7 | 191 |
| JPO-048 | 4.3 | 163 |
| JPO-051 | 5.7 | 222 |
| JPO-058 | 4.2 | 157 |
| JPO-062 | 4.2 | 159 |
| JPO-063 | 5.4 | 107 |
| JPO-064 | 4.9 | 178 |
| JPO-065 | 7.0 | 127 |
| JPO-066 | 6.5 | 178 |
| JPO-069 | 4.8 | 95 |
| JPO-071 | 6.1 | 128 |
| JPO-074 | 6.3 | 108 |
| JPO-081 | 5.5 | 213 |
| JPO-082 | 5.6 | 215 |
| JPO-089 | 6.0 | 171 |
| JPO-090 | 5.5 | 155 |
| JPO-018 | 0.6 | 84 |
| JPO-019 | 0.5 | 86 |
| JPO-044 | 6.3 | 225 |
| JPO-083 | 6.1 | 103 |
| JPO-084 | 4.4 | 66 |
| JPO-099 | 6.8 | 156 |
| JPO-091 | 6.6 | 130 |
| JPO-092 | 6.7 | 113 |
| JPO-093 | 6.8 | 132 |
| JPO-094 | 6.6 | 126 |
| JPO-095 | 6.9 | — |
| JPO-096 | 5.9 | — |
| JPO-097 | 5.2 | — |
| JPO-098 | 5.6 | — |
| JPO-112 | 8.2 | — |
| JPO-114 | 5.2 | 218 |
| JPO-115 | 8.0 | — |
| JPO-108 | 8.5 | — |
| JPO-109 | 7.2 | — |
| JPO-111 | 8.4 | — |
| JPO-124 | 8.0 | 385 |
| JPO-125 | 6.8 | 324 |
| JPO-126 | 6.6 | 268 |
| JPO-127 | 4.9 | 246 |
| JPO-129 | 8.2 | 399 |
| JPO-130 | 5.3 | 278 |
| JPO-131 | 7.9 | 367 |
| JPO-132 | 6.6 | 336 |
| JPO-138 | 6.4 | 125 |
| JPO-133 | 6.1 | 143 |
| JPO-143 | 8.8 | 280 |
| JPO-154 | 7.6 | 252 |
| JPO-155 | 8.3 | 282 |
| JPO-156 | 8.3 | 290 |
| JPO-145 | 8.2 | — |
| JPO-147 | 8.2 | — |
| JPO-150 | 8.2 | — |
| JPO-152 | 8.4 | — |
| JPO-153 | 9.0 | 399 |
| JPO-161 | 6.0 | 200 |
| JPO-165 | 8.9 | 403 |
| JPO-166 | 7.0 | 237 |
| JPO-167 | 9.1 | 387 |
| JPO-168 | 9.3 | 332 |
| JPO-169 | 9.6 | 269 |
| JPO-171 | 9.4 | 255 |
| JPO-172 | 9.9 | 432 |

### Example 7: Baking with Reduced Dosage JPO-172

Breads were baked in a straight dough baking process with a recipe according to Table 4. Different treatments were made according to Table 5, where the performance of JPO-172 was compared with the current top-performing anti-staling commercial baking maltogenic alpha-amylase product, Novamyl® 3D (Novozymes, Denmark). The bread was baked in lidded tins. The ingredients were mixed in a pin mixer into a dough for 1 min at 1^{st} speed followed by 2^{nd} speed to full development. The doughs were allowed to rest for 5 minutes and divided into 645 g dough pieces and rounded. The rounded dough pieces were allowed to rest for 10 min, sheeted and placed in baking tins. The tins with the doughs were proofed to height at 104-109F and 85% relative humidity. For lidded pans the doughs were proofed until the dough is ¾″ from the top. The proofed doughs were baked in a reel oven for 17 minutes at approx. 227 °C. (440 °F).

TABLE 4
Recipe

| Ingredient | Bakers % |
|---|---|
| Flour | 100 |
| Water | 61 |
| Yeast Instant Active Dry sugar tolerant | 2 |
| Sucrose | 8 |
| Salt | 2 |
| SSL | 0.375 |
| GMS-90 | 1 |
| Soybean oil | 3 |
| Ascorbic acid | 0.06 |
| Calcium Propionate | 0.4 |
| Fungamyl ® 4000 SG | 10 ppm |
| Panzea ® BG | 25 ppm |

TABLE 5
Treatments

| | 1 | 2 | 3 | 4 |
|---|---|---|---|---|
| Novamyl ® 3D, | 750 | | | |
| MANU/kg flour | | | | |
| JPO-172, | | 8.2 | 9.8 | 11.5 |
| mgEP/kg flour | | | | |

The bread was packed 2 hours after baking in sealed plastic bags and stored at room temperature until analysis.

The texture of the bread was evaluated with a texture analyzer (TA-XT plus, Stable microsystems, Godalmine, UK). Bread crumb texture properties were characterized by firmness (the same as “hardness” and the opposite of “softness”) and the elasticity of the baked product. A standard method for measuring firmness and elasticity is based on force-deformation of the baked product. A force-deformation of the baked products may be performed with a 40 mm diameter cylindrical probe. The force on the cylindrical probe is recorded as it is pressed down 27% strain on a 25 mm thick bread slice at a deformation speed of 1 mm/second. The probe is then kept in this position for 30 seconds while the force is recorded and then probe returns to its original position.

Firmness (in grams) is defined as the force needed to compress a probe to a 25% strain (corresponding to 6.25 mm compression into a bread crumb slice of 25 mm thickness).

Elasticity (in %) is defined as the force recoded after 30 seconds compression at 27% strain (corresponding to force at time=36.75 s for a bread slice of 25 mm thickness) divided by the force needed to press the probe 6.75 mm into the crumb (corresponding to force at time=6.75 s for a bread slice of 25 mm thickness) times 100.

The results from the texture analysis can be found in Table 6 and Table 7. A fresh bread is soft (low firmness) and elastic. As the bread is stored it becomes more firm and less elastic. The higher dosages of JPO-172 the less firm and the higher elasticity the bread has on day 7-21. However, at these conditions and at these dosages the softest and most elastic was the bread with 750 MANU/kg flour of Novamyl® 3D in the time period of 1-21 days.

TABLE 6
Firmness (g) of bread crumb at different timepoints.

| Treatment | Day 1 | Day 7 | Day 14 | Day 21 |
|---|---|---|---|---|
| Novamyl ® 3D 750 MANU/kg | 122 | 232 | 279 | 348 |
| flour | | | | |
| JPO-172 8.2 mgEP/kg flour | 171 | 327 | 482 | 549 |
| JPO-172 9.8 mgEP/kg flour | 158 | 343 | 466 | 549 |
| JPO-172 11.5 mgEP/kg flour | 147 | 320 | 345 | 464 |

TABLE 7
Elasticity (%) of bread crumb at different timepoints.

| Treatment | Day 1 | Day 7 | Day 14 | Day 21 |
|---|---|---|---|---|
| Novamyl ® 3D 750 MANU/kg | 62 | 59 | 58 | 58 |
| flour | | | | |
| JPO-172 8.2 mgEP/kg flour | 61 | 55 | 52 | 50 |
| JPO-172 9.8 mgEP/kg flour | 61 | 54 | 52 | 49 |
| JPO-172 11.5 mgEP/kg flour | 61 | 55 | 55 | 52 |

### Example 8: Baking with Reduced Dosage JPO-172 at Lower pH

Breads were baked in a straight dough baking process with a recipe according to Table 8. Different treatments were made according to Table 9. The bread was baked in lidded tins. The ingredients were mixed in a pin mixer into a dough for 1 min at 1^{st} speed followed by 2^{nd} speed to full development. The doughs were allowed to rest for 5 minutes and divided into 645 g dough pieces and rounded. The rounded dough pieces were allowed to rest for 10 min, sheeted and placed in baking tins. The tins with the doughs were proofed to height at 104-109F and 85% relative humidity. For lidded pans the doughs were proofed until the dough is ¾″ from the top. The proofed doughs were baked in a reel oven for 17 minutes at approximately 227 °C (440 °F).

TABLE 8
Recipe

| Ingredients | Bakers % |
|---|---|
| Flour | 100 |
| Water | 62 |
| Yeast Instant Active Dry sugar tolerant | 2 |
| Sucrose | 9 |
| Salt | 2 |
| Vinegar (50 grain) | 2 |
| Soybean oil | 3 |
| Ascorbic acid | 0.06 |
| Calcium Propionate | 0.3 |
| Fungamyl ® 4000 SG | 10 ppm |
| Panzea ® BG | 25 ppm |

TABLE 9
Treatments

| | 1 | 2 | 3 | 4 |
|---|---|---|---|---|
| Novamyl ® 3D, | 750 | | | |
| MANU/kg flour | | | | |
| JPO-172, | | 8.2 | 9.8 | 11.5 |
| mgEP/kg flour | | | | |

The bread was packed 2 hours after baking in sealed plastic bags and stored at room temperature until analysis.

The texture analysis was performed as in example 7. The results from the texture analysis can be found in Table 10 and Table 11. A fresh bread is soft (low firmness) and elastic, as the bread is stored it becomes more firm and less elastic in the absence of anti-staling enzymes.

We conclude that addition of 2% vinegar, thereby lowering the dough pH, made it possible to lower the dosage of JPO-172 below 12 mgEP/kg flour, while still providing an anti-staling effect on par with or better than Novamyl® 3D.

TABLE 10
Firmness (g) of bread crumb at different timepoints.

| Treatment | Day 1 | Day 7 | Day 14 | Day 21 |
|---|---|---|---|---|
| Novamyl ® 3D 750 MANU/kg | 183 | 260 | 310 | 325 |
| flour | | | | |
| JPO-172 8.2 mgEP/kg flour | 164 | 324 | 344 | 364 |
| JPO-172 9.8 mgEP/kg flour | 164 | 303 | 316 | 349 |
| JPO-172 11.5 mgEP/kg flour | 145 | 250 | 282 | 305 |

TABLE 11
Elasticity (%) of bread crumb at different timepoints.

| Treatment | Day 1 | Day 7 | Day 14 | Day 21 |
|---|---|---|---|---|
| Novamyl ® 3D 750 MANU/kg | 65.7 | 62.6 | 62.5 | 61.8 |
| flour | | | | |
| JPO-172 8.2 mgEP/kg flour | 65.8 | 60.5 | 60.9 | 60.5 |
| JPO-172 9.8 mgEP/kg flour | 66.3 | 61.4 | 61.9 | 60.9 |
| JPO-172 11.5 mgEP/kg flour | 67.1 | 62.9 | 63.3 | 62.8 |

### Example 9. Activity of JPO-172 as a Function of pH

The pH activity profile is a property which is characteristic for an enzyme and important for its usage in different applications. The pH profile (in the range of pH 2-10) for JPO-172 was determined at 40 °C and 30 minutes incubation.

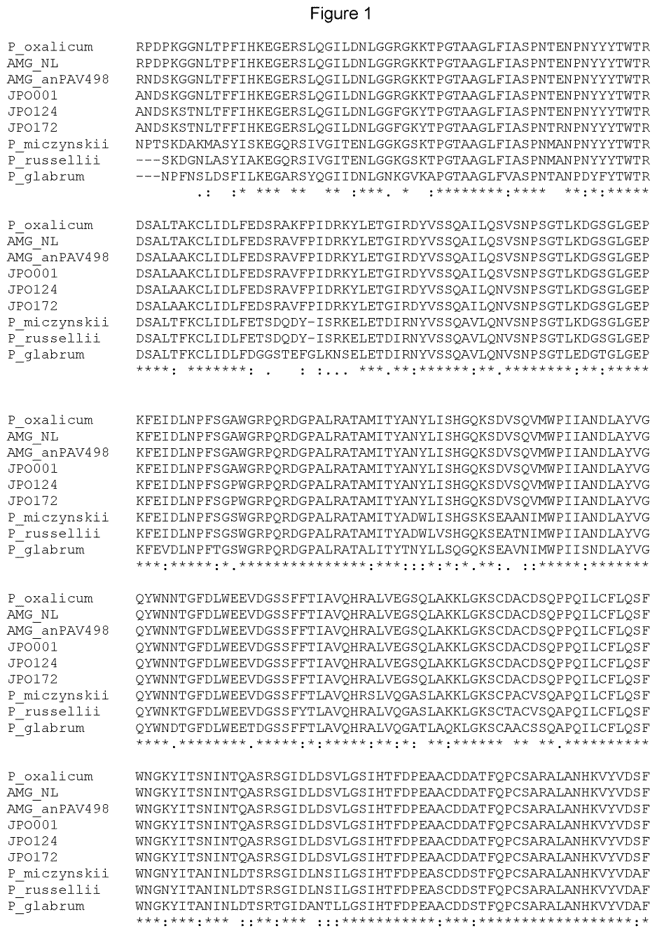

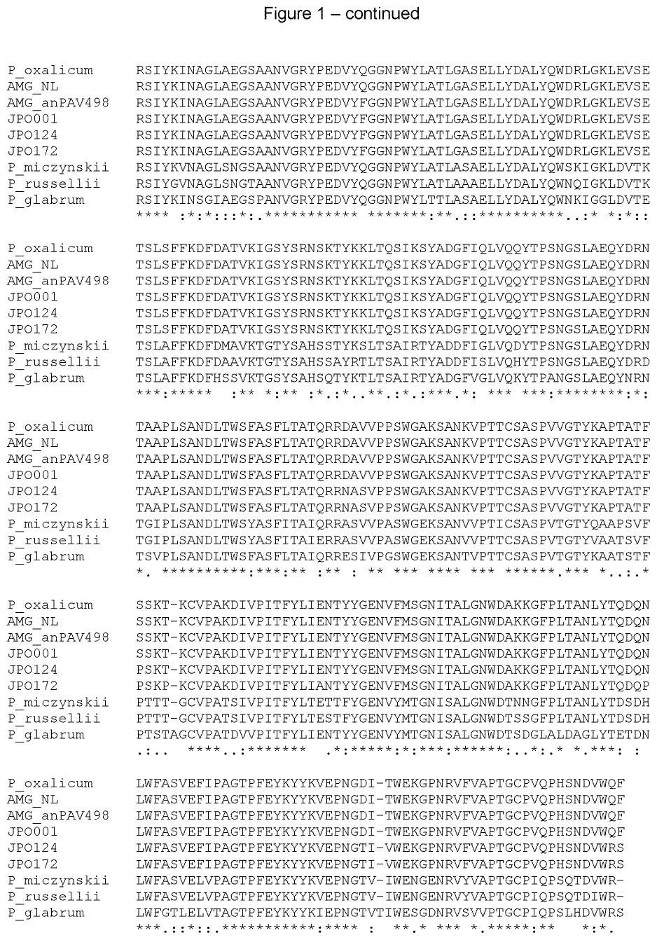

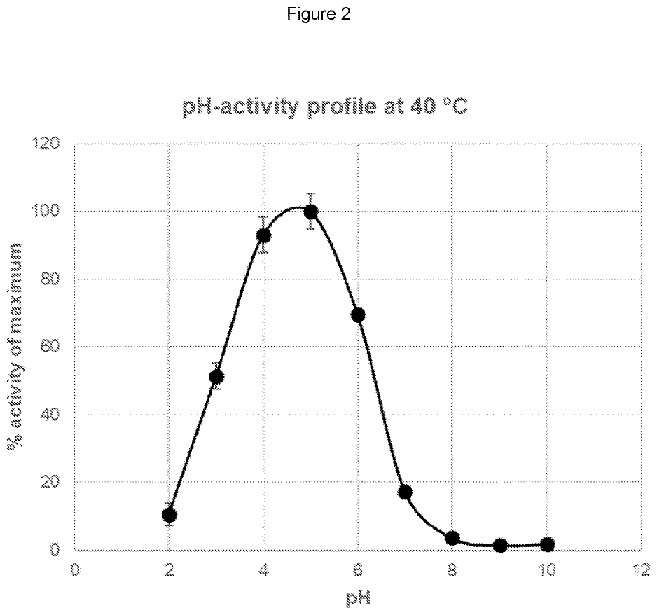

30 mM maltose (CAS number: 6363-53-7) substrate solution was prepared in buffer (0.1 M Acetic acid; 0.1 M MES; 0.1 M HEPES; 0.1 M Glycine) adjusted to pH 2-10 using HCl or NaOH. The pH activity profile was prepared by adding 15 μL diluted enzyme sample (10 ppm, diluted in 20 mM MES, pH 5) or buffer to 135 μL substrate solution in Eppendorf tubes. The pH of the reaction mixtures was determined. The mixture was incubated for 30 minutes at 40 °C and 800 rpm. After 30 minutes, the reaction was terminated by adding 16 μL 0.5 M NaOH and placed on ice. The reaction mixture was diluted 10 times with 20 mM MES, pH 5 in a micro titer plate. 40 μL of the diluted solution was mixed in a new micro titer plate with 160 μL GOD-POD reaction mixture and incubated for 30 minutes in absence of light. After incubation, the absorbance at 420 nm was measured. An average of reaction mixtures was subtracted an average of blank samples and is presented in FIG. 2.

The pH-activity profile is shown in FIG. 2. All data points are averages of four measurements and are given relative pH 5 which is set to 100%. In the profile, it can be seen that pH optimum is around pH 5 and that approximately 80% activity is observed from approximately pH 4 to 6.

1. A method of producing a baked or par-baked product, said method comprising:
a) providing a dough comprising a mature thermostable variant of a parent glucoamylase at least 70% identical to SEQ ID NO:1, SEQ ID NO:6, SEQ ID NO:7; SEQ ID NO:8 or SEQ ID NO:10 added in an amount of 0.01-12.40 mg enzyme protein (mgEP) per kg flour, wherein said dough has a pH value in the range of 3.0-6.5; and
b) baking or par-baking the dough to produce a baked or par-baked product.

2. The method according to claim 1, wherein the baked or par-baked product is a type of bread, preferably a pan bread, toast bread, open bread, buns, Fino bread, Hammam bread, Samoli bread, baguettes, brioche, hamburger buns, rolls, brown bread, whole meal bread, rich bread, bran bread, flat bread, tortilla or biscuit, cake or a patisserie.

3. The method according to claim 1, wherein the parent glucoamylase is from a species of Penicillium.

4. The method according to claim 1, wherein the mature variant comprises at least one amino acid modification in one or more or all of the positions corresponding to positions 1, 2, 4, 6, 7, 11, 31, 34, 65, 79, 103, 132, 327, 445, 447, 481, 566, 568, 594 and 595 in SEQ ID NO:1.

5. The method according to claim 4, wherein the at least one amino acid modification comprises a substitution in one or more or all of the positions corresponding to positions 1, 2, 4, 11, 65, 79 and 327 in SEQ ID NO:1.

6. The method according to claim 4, wherein the at least one amino acid modification comprises a substitution in one or more or all of the positions corresponding to positions 1, 6, 7, 31, 34, 79, 103, 132, 445, 447, 481, 566, 568, 594 and 595 in SEQ ID NO:1.

7. The method according to claim 1, wherein the at least one amino acid modification comprises a substitution in one or more or all of the positions corresponding to positions 1, 6, 7, 31, 34, 50, 79, 103, 132, 445, 447, 481, 484, 501, 539, 566, 568, 594 and 595 in SEQ ID NO:1.

8. The method according to claim 1, wherein the mature thermostable variant has a thermostability improvement (Td) over its parent of at least 3 °C.

9. The method according to claim 1, wherein the mature thermostable variant has a relative activity at 91 °C of at least 150.

10. The method according to claim 1, wherein no emulsifier is added to the dough.

11. The method according to claim 1, wherein the baked or par-baked product after final bake-off has a reduced initial firmness and/or an increased initial elasticity, and/or a reduced increase in firmness and/or a higher elasticity after 1, 7 or 14 days, when cooled to room temperature, packed in a sealed container and stored at room temperature until analysis, compared to a control made without any added glucoamylase.

12. The method according to claim 1, wherein the baked or par-baked product after final bake-off has at least the same sweetness as a control product made with double the amount of the mature glucoamylase the amino acid sequence of which is shown in SEQ ID NO:10.

13. The method according to claim 1, wherein the dough also comprises one or more additional enzyme selected from the group consisting of a alpha-amylase, maltogenic amylase, raw-starch degrading alpha-amylase, beta amylase, aminopeptidase, carboxypeptidase, catalase, cellulytic enzyme, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, proteolytic enzyme, ribonuclease, transglutaminase, and xylanase; wherein the one or more additional enzyme is comprised in an amount of 0.01-1,000 mg enzyme protein (mgEP) per kg flour.

14. The method according to claim 1, wherein the parent glucoamylase is from Penicillium oxicalum, Penicillium miczynskii, Penicillium russellii or Penicillium glabrum.

15. The method according to claim 4, wherein the at least one amino acid modification comprises a substitution in one or more or all of the positions corresponding to R1A, P2N, P4S, P11F, T65A, K79V and Q327F in SEQ ID NO:1.

16. The method according to claim 4, wherein the at least one amino acid modification comprises a substitution in one or more or all of the positions corresponding to R1A, G6S, G7T, R31F, K34Y, K79V, S103N, A132P, D445N, V447S, S481P, D566T, T568V, Q594R and F595S in SEQ ID NO:1.

17. The method according to claim 1, wherein the at least one amino acid modification comprises a substitution in one or more or all of the positions corresponding to R1A, G6S, G7T, R31F, K34Y, E50R, K79V, S103N, A132P, D445N, V447S, S481P, T484P, E501A, N539P, D566T, T568V, Q594R and F595S in SEQ ID NO:1.
